(19) United States
(12) Patent Application Publication
(10) Pub. No.: US 2005/0054748 A1
(43) Pub. Date: Mar. 10, 2005

(54) Dental composition

(76) Inventors: Joachim E. Klee, Radolfzell (DE); Uwe Walz, Konstanz (DE); Rolf Mulhaupt, Freiburg (DE)

Correspondence Address: Douglas J. Hura, Esquire;DENTSPLY International Inc., 570 West College Avenue, P.O. Box 872, York, PA, 17405-0872, US

(21) Appl. No.: 10/943,355

(22) Filed: Sep. 17, 2004

Related U.S. Application Data
(63) Continuation of application No. 10/156,903, filed on May 29, 2002, now abandoned.
Application No. 10/156,903 is a continuation of application No. 09/458,848, filed on Dec. 10, 1999, now abandoned.
(60) Provisional application No. 60/111,864, filed on Dec. 11, 1998.

(51) Int. Cl. A61K 6/08

(52) U.S. Cl. 523/116

(57) ABSTRACT

A dental composition including a mono- and/or a poly-cyanoacrylate, a polymerizable monomer, a stabilizer, an initiator, and optionally, pigments and a filling material.

### RELATED APPLICATIONS

This is a continuation of U.S. patent application Ser. No. 10/156,903 filed May 29, 2002 (Case KON-118 CPA CON), which is a continuation of U.S. patent application Ser. No. 09/458,848 filed Oct. 16, 2001 (Case KON-118-CPA Abandoned), which is a Continued Prosecution Application (CPA) of U.S. patent application Ser. No. 09/458,848 filed Dec. 10, 1999 (Case KON-118 Abandoned), which claims benefit of U.S. Provisional Patent Application Ser. No. 60/111,864 filed Dec. 11, 1998 (Case KON-118).

### TECHNICAL BACKGROUND

Described is a dental composition comprising mono- and/or a polycyanoacrylate, a polymerizable monomer, a stabilizer, an initiator, pigments and a filling material. The invented dental composition is usable as dental filling material, dental cement, dental sealer or as dental adhesive.

### BACKGROUND OF THE INVENTION

Dental cements that are available on the market are Zn-phosphate cements, glass ionomer cements, self-curing adhesives on basis of composite resins or a relatively new class of compomer cements. All these cements require a mixing of two components to reach a solid product. Frequently, powder and liquid or two pastes were applied. However, the solidification is a process that is relatively independent of the applicator. That means after mixing the components the process of free-radical polymerization or acid-base reaction takes place.

Under this point of view light-curing cements are advantageously, because they polymerize when irradiated with visible light. However, only for a part of the applications light polymerizable cements are usable, e.g. for composite or ceramic inlays, onlays or crowns. Metallic and metal fused porcelain restorations are only adherable by using of self-curing cements.

Consequently, an one component self-curing cement should represent an great advantage. This cement should by a command-setting material that is applicable under metallic or highly-opaque materials, too. One possibility to realize this aim is the application of mono- and polycyanoacryaltes due to their possibility to polymerized in presence of water and amines or other anionic initiators. The anionic polymerization of cyanoacrylates seemed to be advantageous due to the complete polymerization of the material. A smearlayer comparable to the oxygen inhibited layer of free-radical polymerizations is completely missing.

Synthesis and properties of modified cyanoacrylates as well as their anionic polymerization were investigated some years ago (U.S. Pat. No. 3,316,227; N. N. Trofimov et al. Zh. Vses. Khim. O-va. 19 (1974) 473; Z. Denchev et al., J. Appl. Polym. Sci. 42 (1991), 2933).

Recently, the application of cyanoacrylates for electric and electronic applications (DE-96-19640202, WO 9814526), as fast-curing adhesives for metals (JP 59047272, JP 59049099; V. Vijayalakshim et al., J. Appl. Polym. Sci. 49 (1993), 1387), as waterproof instant bonding agents (JP 57164173) and also as surgical adhesives (FR 2010589) was described.

Butylcyanoacrylate is used as a glue for fixation of bone fragments (M. A. Shermak et al., Plast Reconstr Surg 1998 August;102 (2):319-24). Isopropyl cyanoacrylate is applied as root canal cement (E. L. Jacobson et al., J. Endodontics 16 (1990) 516). However, monocyanoacrylates are limited concerning mechanical stability and due to moisture sensitivity. Furthermore, they are disadvantageous due to solubility of the linear polymers.

### DESCRIPTION OF THE INVENTION

Described is a dental composition comprising at least a mono- and/or a polycyanoacrylate, a polymerizable monomer, a stabilizer, an initiator, pigments and a filling material. The mono- and polycyanoacrylates are characterized by the following structure: 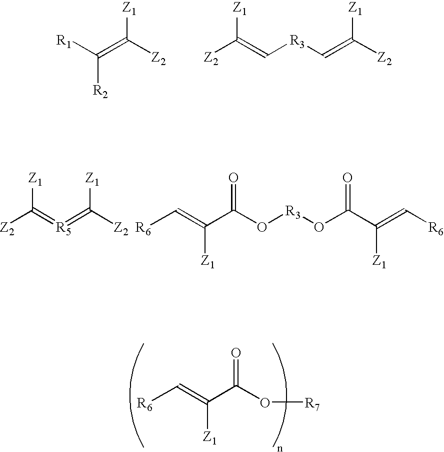
wherein
Z₁ denotes CN, COOR₄, COR₄, NO₂
Z₂ denotes CN, COOR₄, COR₄, NO₂
R₁ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₂ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₃ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,
R₄ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₅ denotes a difunctional substituted or unsubstituted C₁ to C₁₈ alkylene, C₅ to C₁₈ substituted or unsubstituted cycloalkylene,
R₆ denotes a substituted or unsubstituted C₁ to C₁₈ alkylene, C₅ to C₁₈ substituted or unsubstituted cycloalkylene, substituted or unsubstituted C₅ to C₁₈ arylene or heteroarylene.
R₇ denotes a polyfunctional substituted or unsubstituted alkylene, substituted or unsubstituted cycloalkylene, substituted or unsubstituted arylene or heteroarylene, selected from the group

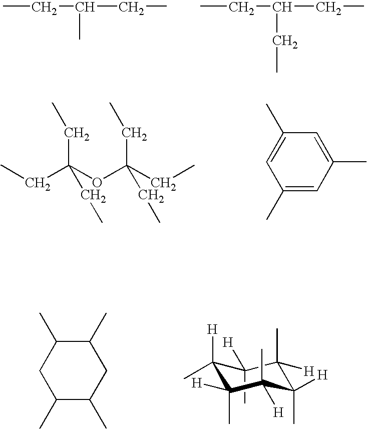

Preferably, the mono- and polycyanoacrylates are characterized by the following structures: 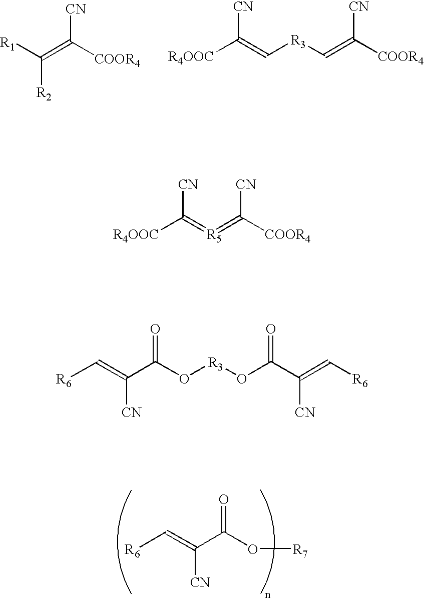
wherein
R₁ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₂ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₃ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,
R₄ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₅ denotes a difunctional substituted or unsubstituted C₁ to C₁₈ alkylene, C₅ to C₁₈ substituted or unsubstituted cycloalkylene,
R₆ denotes a substituted or unsubstituted C₁ to C₁₈ alkylene, C₅ to C₁₈ substituted or unsubstituted cycloalkylene, substituted or unsubstituted C₅ to C₁₈ arylene or heteroarylene.
R₇ denotes a polyfunctional substituted or unsubstituted alkylene, substituted or unsubstituted cycloalkylene, substituted or unsubstituted arylene or heteroarylene, selected from the group

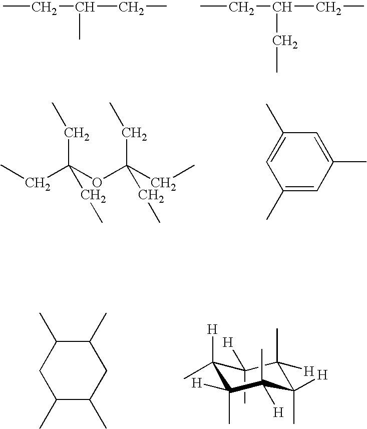

Most preferably the following mono- and polycyanoacrylate are usable in a dental composition:

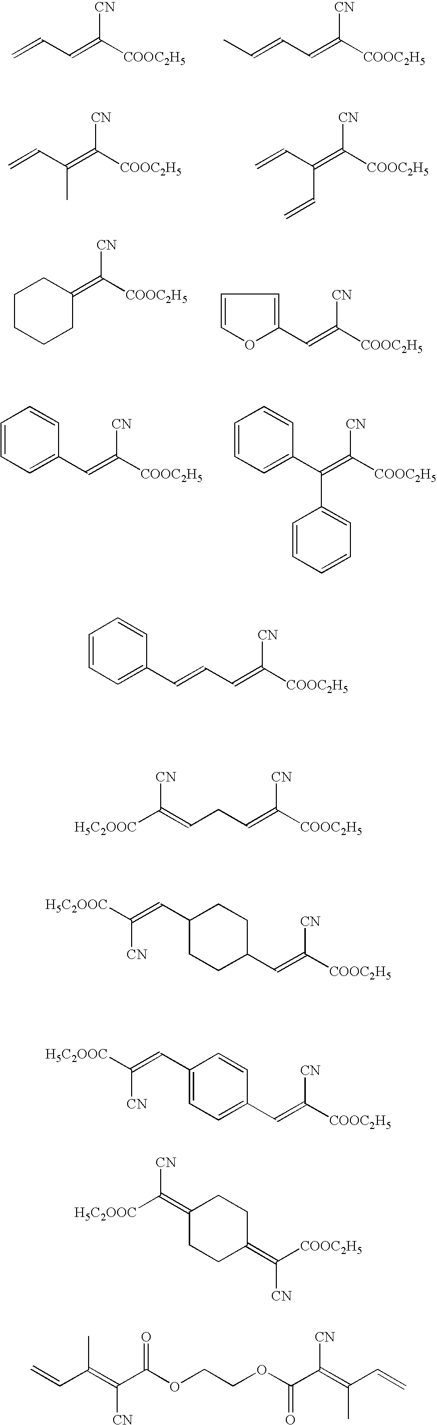

As polymerizable monomers are used mono- and polyfunctional (meth)-acrylates, such as a polyalkylenoxide di- and poly(meth)acrylate, an urethane di- and poly(meth) acrylate, a vinyl-, vinylen- or vinyliden-, acrylate- or methacrylate substituted spiroorthoester, a spiroorthocarbonate or a bicyloorthoester; preferably were used diethylenglycol dimethacrylate, triethylenglycol dimethacrylate, 3,(4),8,(9)-dimethacryloyloxymethyl tricyclo-decane, dioxolan bismethacrylate, glycerol trimethacrylate, furfuryl methacrylate in a content of 5 to 80 wt-%.

The polymerization initiator for the dental composition is a thermal initiator or a photoinitiator and/or an anionic polymerization initiator. Preferably, the polymerization of the invented mono- and polycyanoacrylates is initiated by free-radical and anionic polymerization initiators or only by an anionic polymerization initiator. Most preferably, water acts as anionic polymerization initiator. Furthermore, the well-known Reinecke salt (K⁺Cr(NH₃)₂(NCS)₄⁻ (C. Kutsl et al, Macromolecules 24 (1991) 6872) and group IV metal carbonyl pyridine complexes (R. B. Paul et al. Polymer 38 (1997) 2011) are usable as photoinitiators for photoinduced anionic polymerization.

The monomers are stabilized by using radical absorbing monomer such as hydrochinon monomethylether, hydrochinondimethylether, BHT. Mono- and polycyanoacrylates are stabilized by using of acids such as phosphoric acid, arylphosphonic acid, SO₂, p-toluensulfonic acid.

The invented dental composition comprises as filing materials inorganic and/or organic fillers.

The invented dental composition is usable as dental filling material, dental cement, dental sealer or as dental adhesive.

For example 2,6-Dicyano-hepta-2,5-dien-dicarboxylic acid diethylester and a barium silicate glass were mixed homogeneously. When applying this composite under humid conditions a spontaneous anionic polymerization takes place.

Mono- and polycyanoacrylates are usable in a dental compositions such as in restoratives, adhesives, bases and liners, root canal sealers and for others.

Furthermore, the invented mono- and polycyanoacrylates are usable in electronics, microelectronics as industrial adhesives or for medical applications.

### EXAMPLE 1

### 2-Cyano-2,4-pentadienoic acid butylester (CPABE)

In a 250-ml bottle equipped with a condenser a mixture of 29.73 g ZnCl₂ and 70 ml Dioxan was refluxed under stirring. To the cooled mixture 50.36 g (0.357 mol) cyanoacetic acid butyl ester, 25.00 g (0.446 mol) acrolein were added and stirred for 65 hours at room-temperature. Then the mixture was poured in 300 ml 5% age HCl. The crude product was filtered off, dissolved in 300 ml CH₂Cl₂ and dried over NaSO₄. Then CH₂Cl₂ was removed and the cyanoacrylate was distilled product was recrystallized.

Yield: 57.46 g (89.9%), bp. 93-96 (0.5)°C., n₂₀^{D}=1.4960, η_{23° C.}=0.354±0.019 Pa*s C₁₀H₁₃NO₂, 179.21

IR: 2227 (CN), 1728 (CO), 1617/1583 (C═C) cm⁻¹

¹³C NMR (CDCl₃): 162.0 (6), 155.1 (3), 133.8 (2), 113.8 (5), 132.0 (1), 107.5 (4), 66.3 (7), 30.9 (8), 19.0 (9), 13.6 (10)

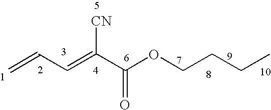

### EXAMPLE 2

### 3-(2-Furanyl)-2-cyano-prop-2-en carboxylic acid ethylester (FCPCE)

In a 250-ml bottle equipped with a condenser a mixture of 25.000 g ZnCl₂ and 100 ml Dioxan was refluxed under stirring. To the cooled mixture 36.030 g (0.375 mol) furfural and 33.935 g (0.300 mol) cyano acidic acid were added and stirred for four hours at room-temperature. Then the mixture was poured in 400 ml 5% age HCl. The crude product was filtered off, dissolved in THF and dried over NaSO₄. After removing THF the product was recrystallized.

Yield: 21.37 g (32.16%)

IR: 3055, 2985 (CH₃/CH₂), 2225 (CN), 1722 (CO), 1618/1604 (C═C), 1465 (CH₃), 1260 (C—O—C)

¹³C-NMR: 14.1 (1), 62.5 (2), 162.5 (3), 98.7 (4), 115.3 (5), 148.7 (6), 148.2 (7), 113.8 (8), 121.6 (9), 139.4 (10)

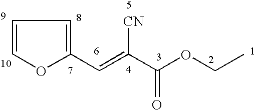

### EXAMPLE 3

### 2-Cyano-2,4-hexadienoic acid ethyl ester (CHAEE)

In a 250-ml bottle equipped with a condenser a mixture of 25.04 g ZnCl₂ and 100 ml Dioxan was refluxed under stirring. To the cooled mixture 36.030 g (0.375 mol) crotonaldehyde and 33.950 g (0.300 mol) cyano acidic acid were added and stirred for four hours at room-temperature. Then the mixture was poured in 400 ml 5% age HCl. The crude product was filtered off, dissolved in THF and dried over NaSO₄. After removing THF the product was recrystallized.

Yield: 157.5 g (92.8%), mp. 56-58° C.

IR: 2227.7 cm⁻¹ (CN), 1725.4 cm⁻¹ (CO), 1634.5/1586.9 cm⁻¹ (C═C)

¹³C-NMR (CDCl₃): 162.1 (7), 155.6 (4), 149.7 (3), 127.7 (2), 114.1 (6), 103.3 (5), 62.1 (8), 19.3 (1), 14.0 (9)

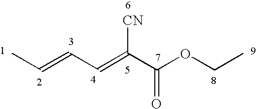

### EXAMPLE 4

### 2-Cyano-2,4-hexadienoic acid butylester (CHABE)

In a 250-ml bottle equipped with a condenser a mixture of 29.39 g ZnCl₂ and 100 ml Dioxan was refluxed under stirring. To the cooled mixture 30.90 g (440.86 mmol) crotonaldehyde and 49.79 g (352.71 mmol) cyano acidic acid were added and stirred for four hours at room-temperature. Then the mixture was poured in 400 ml 5% age HCl. The aqueous solution was extracted twice with CH₂Cl₂ and the extracts were dryed over NaSO₄. Then the solvent was evaporated and the the crude product was distilled in vacuum.

Yield: 59.86 g (87.83%), bp. 101-103° C./0.2 mbar, n₂₀^{D}=1.5104, η_{23° C.}=0.337±0.017 Pa*s C₁₁H₁₅NO₂, 193.24

IR: 2226 (CN), 1728 (CO), 1633/1587 (C═C) cm⁻¹

¹³C-NMR: 155.6 (1), 103.5 (2), 114.8 (3), 162.3 (4), 66.0 (5), 30.4 (6), 18.9 (7), 13.5 (8), 149.6 (9), 127.8 (10), 19.4 (11) 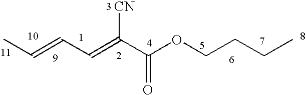
Ethylenglykol-bis(cyano acidic acid ethyl ester) (EGBCE)

In a 500-ml-bottle equipped with water separator and refluxer 32.000 g (0.516 mol) ethyleneglycol and 87.719 g (1.031 mol) cyano acidic acid were dissolved in 200 ml toluene. After addition of 1.197 g (0.007 mol) p-toluene sulfonic acid the mixture was refluxed for six hours or until the calculated amount of water was separated. Then the solvent was removed. The crude product was dissolved in CH₂Cl₂, extracted twice with 50 ml water and dried over NaSO₄. After removing the solvent the product was distilled in vacuum.

Yield: 59.63 g (59.0%), bp.₁₀=55° C., n₂₀^{D}=1.4603, η=0.45±0.02 Pa*s C₈H₈N₂O₄, 196.16

IR: 2973/2935 (CH₃/CH₂), 2266 (CN), 1747 (CO), 1182 cm⁻¹ (C—O—C)

¹H-NMR (CDCl₃): 3.51 (CH₂—CN), 4.40 (O—CH₂)

¹³C-NMR (CDCl₃): 162.9 (3), 112.7 (1), 63.6 (4), 24.6 (2)

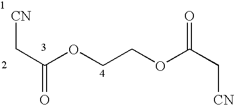

### EXAMPLE 5

### Bis-(2-Cyano-2,4-hexadienoic acid)-1,2-ethanediyl ester (EGBCS)

In a 250-ml bottle equipped with a condenser a mixture of 12.666 g ZnCl₂ and 50 ml Dioxan was refluxed under stirring. To the cooled mixture 22.460 g (0.320 mol) crotonaldehyde and 25.143 g (0.128 mol) EGBCE were added and stirred for four hours at room temperature. Then the mixture was poured in 400 ml 5% age HCl. The crude product was separated, dissolved in CH₂Cl₂ and dried over NaSO₄. After removing THF the product was distilled in vacuum.

Yield: 26.83 g (69.70% of. th.), mp. 133-136° C. C₁₆H₁₆N₂O₄, 300.31

IR: 2972/2933 (CH₃/CH₂), 2227 (CN), 1753 (CO), 1633/1582 (C═C), 1247 cm⁻¹ (C—O—C)

¹³C-NMR: 162.1 (7), 156.5 (4), 150.7 (3), 127.9 (2), 113.9 (6), 102.7 (5), 63.1 (8), 19.5 (1)

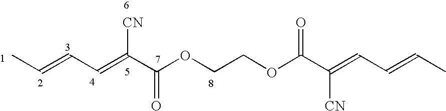

### EXAMPLE 6

### Diethylenglykol-bis(cyano acidic acid ethyl ester) (DEGBCE)

In a 500-ml-bottle equipped with water separator and refluxer 45.000 g (0.424 mol) diethyleneglycol and 79.353 g (0.933 mol) cyano acidic acid were dissolved in 100 ml toluene. After addition of 1.244 g (0.007 mol) p-toluene sulfonic acid the mixture was refluxed for six hours or until the calculated amount of water was separated. Then the solvent was removed. The crude product was dissolved in CH₂Cl₂, extracted twice with 50 ml water and dried over NaSO₄. After removing the solvent the product was distilled in vacuum.

Yield: 84.94 g (84.4% of th.), mp.=37-42.5° C., n D=1.4648, η=0.52±0.03 Pa*s C₁₀H₁₂N₂O₅ 240.22

IR: 2969/2935 (CH₃/CH₂), 2264 (CN), 1739 (CO), 1122 cm⁻¹ (C—O—C)

¹H-NMR (CDCl₃): 3.51 (CH₂—CN),4.33 (COO—CH₂), 3.69 (O—CH₂)

¹³C-NMR (CDCl₃): 113.2 (1), 24.5 (2), 163.1 (3), 65.2 (4), 68.3 (5) 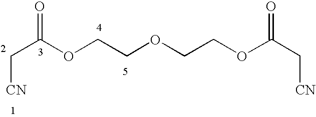
Bis-(2-Cyano-2,4-hexadienoic acid)-1,2-bis(ethanediyl oxy) ester (DEGBCS)

DEGBCA was prepared according the same procedure described in example 7.

Yield: 20.5 g (75.4% of th.), C₁₈H₂₀N₂O₅, 344.37 g/mol

¹³C-NMR: 162.1 (7), 156.0 (4), 150.3 (3), 127.8 (2), 114.0 (6), 103.0 (5), 68.6 (9), 65.0 (8), 19.4 (1)

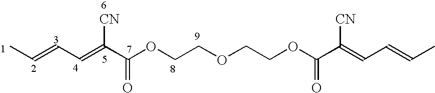

### EXAMPLE 7

### 2,6-Dicyano-hepta-2,5-dien-dicarbonsäturediethylester (DCHDE)

In a bottle equipped with a water separator and a condenser a mixture of 113.11 g (1.00 mol) cyanoacetic acid ethyl ester, 36.03 g (0.50 mol) malondialdehyde, 6.03 g (0.05 mol) piperidiniumhydrochlorid and 6.01 g (0.10 mol) acidic acid was dissolved in 150 ml benzene and refluxed for until the end of water separation (2 to 6 hours). The cold reaction mixture was extracted four times with half-saturated sodium chloride solution and dried over sodium sulfate. Then the benzene was distilled off and the cyanoacrylate was distilled.

Yield: 118 g (90% of th.) C₁₃H₁₄N₂O₄, 262.27

IR: 2200 cm⁻¹ (CN)

¹³C NMR: 165.0 (6), 158.5 (3), 117.2 (5), 103.3 (4), 59.1 (7), 20.5 (2), 13.7 (8)

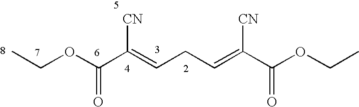

### EXAMPLE 8

### 2-Cyano-2,4-hexadienoic acid (2-Cyano-sorbic acid, 2-CHA)

In a 1-1-three-necked bottle equipped with a stirrer, a dropping funnel and a condenser were dissolved 133.49 g (1.569 mol) cyanoacetic acid in 200 ml Ethanol. To this solution 62.76 g sodium hydroxide dissolved in 65 ml water were added under stirring and cooling up to a pH of 12. Thereafter a solution of 100.00 g (1.427 mol) Crotonaldehyde dissolved in 200 Ethanol was added drop whise under cooling between 5 to 10° C. The complete reaction mixture then was reacted for 5 days at 40° C. The reaction mixture was acidified by adding 154.65 ml HCl conz., 37%. Yellow crystals were obtained by recrystallization from water.

Yield: 80.70 g (41.2% of th.), mp.=150-156° C., C₇H₇NO₂ 137.13

IR: 3414/2594 (COOH), 3004/2974 (CH₃), 2226 (CN), 1654 (CO), 1635/1585 (C═C)

¹³C-NMR (CDCl₃): 163.5 (1), 104.4 (2), 156.5 (3), 115.0 (4), 151.2 (5), 128.3 (6), 19.4 (7) 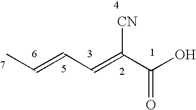
Bis-(2-Cyano-2,4-hexadienoic acid)-3,(4),8,(9)-dimethylene tricyclo-5.2.1.0 ^{2,6}-decane diyl oxy) ester (TCDCS)

In a 500-ml-three-necked bottle equipped with a stirrer, a dropping funnel and a condenser were dissolved 18.20 g (92.72 mmol) Bis-(hydroxymethyl)-tricyclo-5.2.1.0 ^{2,6}-decan and 25.43 g (185.45 mmol) 2-Cyano-2,4-hexadienoic acid in 160 ml Aceton. To this solution was added drop whise under stirring at 0 to 5° C. a solution of 38.26 g (185.45 mmol) N,N′-Dicyclohexylcarbodiimd in 50 ml Aceton. Thereafter the mixture was stirred for 15 minutes at 0° C. and 22 hours at room temperature. The precipitate was filtered off, 0.066 g BHT were added and the solvent was removed by distillation.

Yield: 37.40 g (92.82% of th.) C₂₆H₃₀N₂O₄, 434.54

IR: 2225 (CN), 1714 (CO), 1646/1587 (C═C) cm⁻¹

¹³C NMR: 162.4 (4), 155.8 (1), 149.8 (16), 127.9 (17), 114.1 (3), 103.4 (2), 70.3/69.3 (5), 19.4 (18) and signals of the TCD residue between 24.5 and 49.6 ppm

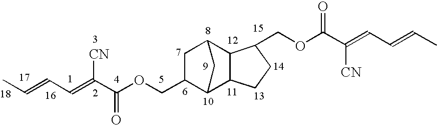

### EXAMPLE 9

### 2,2-Bis-[p-(2-(2-Cyano-2,4-hexadienoyl)-oxypropoxy)-phenyl]-propane (BABCS)

In a 500-ml-three-necked bottle equipped with a stirrer, a dropping funnel and a condenser were dissolved 50.00 g (145.16 mmol) 2,2-Bis-[4-(2-hydroxypropoxy)-phenyl]-propan and 39.81 g (290.32 mmol) 2-Cyano-2,4-hexadienoic acid in 250 ml Aceton. To this solution was added drop whise under stirring at 0 to 5° C. a solution of 59.90 g (290.32 mmol) N,N′-Dicyclohexylcarbodiimd in 80 ml Aceton. Thereafter the mixture was stirred for 15 minutes at 0° C. and 22 hours at room temperature. The precipitate was filtered off, 0.051 g BHT were added and the solvent was removed by distillation.

Yield: 48.48 g (87.70% of th.) C₃₅H₃₈N₂O₆, 582.7 g/mol

Application Example 1—Dental Cement

1.008 g (3.357 mmol) Bis-(2-Cyano-2,4-hexadienoic acid)-1,2-ethanediyl ester (EGBCS) prepared according example 4, 1.001 g (8.000 mmol) Ethylcyanoacrylate and 2.009 g TPH glass were mixed homogeneously.

Compressive strength of samples that were polymerized after initiation with N,N-Dimethyl benzylamine is 65.9±8.3 MPa.

Application Example 2—Dental Cement

1.250 g (5.216 mmol) 2-Cyano-2,4-hexadienoic acid butylester (CHABE) prepared according example 1, were mixed homogeneously with 2.232 g TPH glass.

Compressive strength of samples that were polymerized after initiation with N,N-Dimethyl benzylamine is 57.3±6.1 MPa.

Application Example 3—Dental Cement

1.852 g (33.375% w/w) 2-Cyano-hexadienoic acid ethylester prepared according example 1, were mixed homogeneously with 3.697 g (66.625% w/w) TPH glass.

Compressive strength of samples that were polymerized for 30 minutes at 60° C. after initiation with Trimethylamine is 46.4±6.3 MPa.

1. A dental composition comprising at least a mono- and/or a polycyanoacrylate, a polymerizable monomer, a stabilizer, an initiator, pigments and a filling material.

2. A dental composition of claims 1 comprising at least a mono- and/or a polycyanoacrylate, a polymerizable monomer, a stabilizer, pigments and a filling material.

3. A dental composition of claim 1 wherein said mono- or polycyanoacrylate is characterized by the following structure: 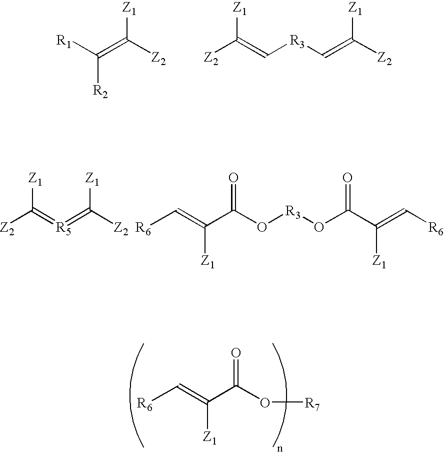
wherein
Z₁ denotes CN, COOR₄, COR₄, NO₂
Z₂ denotes CN, COOR₄, COR₄, NO₂
R₁ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₂ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₃ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,
R₄ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₅ denotes a difunctional substituted or unsubstituted C₁ to C₁₈ alkylene, C₅ to C₁₈ substituted or unsubstituted cycloalkylene,
R₆ denotes a substituted or unsubstituted C₁ to C₁₈ alkylene, C₅ to C₁₈ substituted or unsubstituted cycloalkylene, substituted or unsubstituted C₅ to C₁₈ arylene or heteroarylene.
R₇ denotes a polyfunctional substituted or unsubstituted alkylene, substituted or unsubstituted cycloalkylene, substituted or unsubstituted arylene or heteroarylene, selected from the group

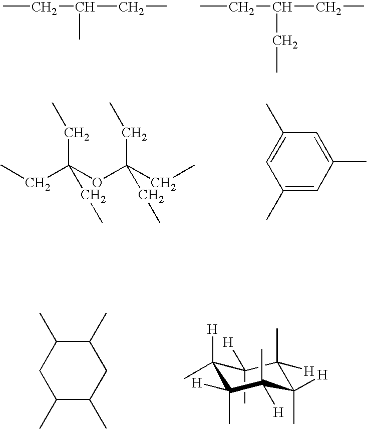

4. A dental composition of claim 1 wherein said mono- or polycyanoacrylate is characterized by the following structure: 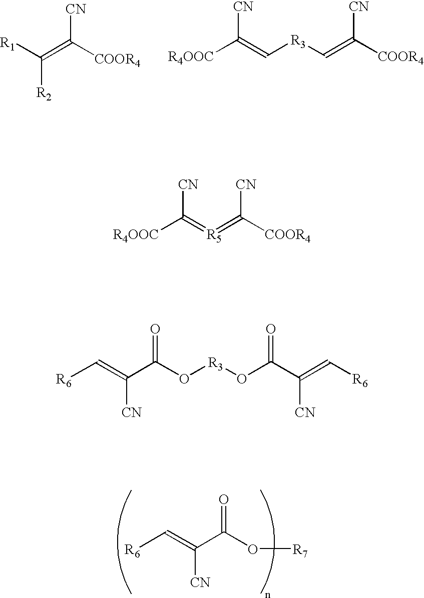
wherein
R₁ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₂ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₃ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,
R₄ denotes hydrogen, or a substituted or unsubstituted C₁ to C₁₈ alkylene,
C₅ to C₁₈ substituted or unsubstituted cycloalkylene, substituted or unsubstituted C₅ to C₁₈ arylene or heteroarylene,
R₅ denotes a difunctional substituted or unsubstituted C₁ to C₁₈ alkylene, C₅ to C₁₈ substituted or unsubstituted cycloalkylene,
R₆ denotes a substituted or unsubstituted C₁ to C₁₈ alkylene, C₅ to C₁₈ substituted or unsubstituted cycloalkylene, substituted or unsubstituted C₅ to C₁₈ arylene or heteroarylene.
R₇ denotes a polyfunctional substituted or unsubstituted alkylene, substituted or unsubstituted cycloalkylene, substituted or unsubstituted arylene or heteroarylene, selected from the group

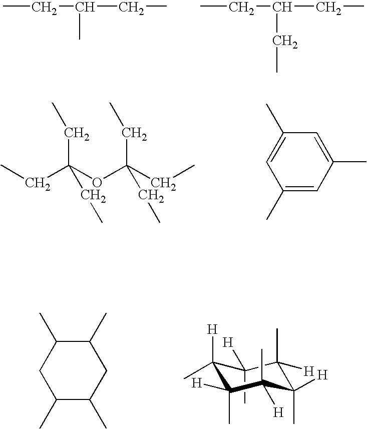

5. A dental composition of claim 1 wherein said monocyanoacrylate is selected from the following group

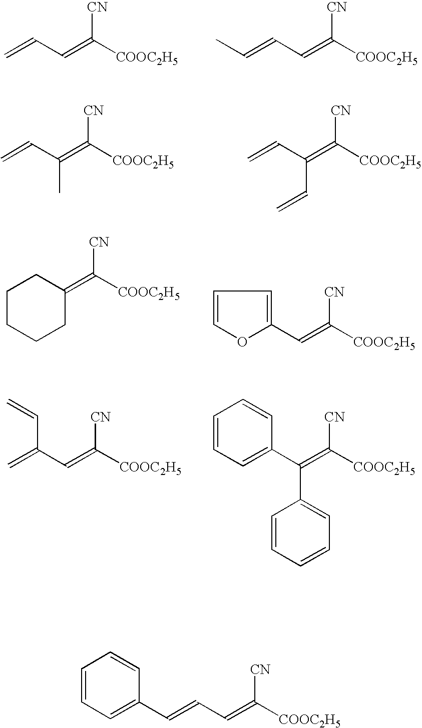

6. A dental composition of claim 1 wherein said polycyanoacrylate is selected from the following group

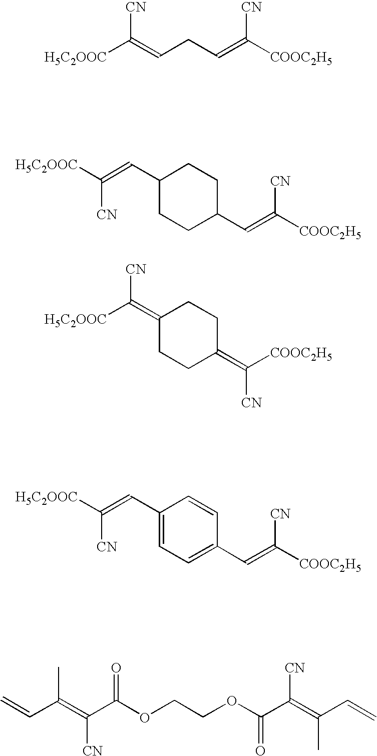

7. A dental composition of claim 1 wherein said polymerizable monomer is a mono- and polyfunctional (meth)-acrylate, such as a polyalkylenoxide di- and poly-(meth)acrylate, an urethane di- and poly(meth) acrylate, a vinyl-, vinylen- or vinyliden-, acrylate- or methacrylate substituted spiroorthoester, a spiroorthocarbonate or a bicylo-orthoester; preferably were used diethylenglycol dimethacrylate, triethylenglycol dimethacrylate, 3,(4),8,(9)-dimethacryloyloxymethyltricyclodecane, dioxolan bismethacrylate, glycerol trimethacrylate, furfuryl methacrylate in a content of 5 to 80 wt-%.

8. A dental composition of claim 1 wherein said polymerization initiator is a thermal initiator, a redox-initiator or a photo initiator and/or a an anionic initiator.

9. A dental composition of claim 1 wherein said anionic initiator is a photoinitiator Isuch as the Reinecke salt (K⁺ Cr(NH₃)₂(NCS)₄⁻ or group IV metal carbonyl pyridine complexes.

10. A dental composition of claim 8 wherein said anionic initiator is water.

11. A dental composition of claim 1 wherein said filler is an inorganic filler and/or an organic filler.

12. A dental composition of claim 1 wherein said stabilizer is a radical absorbing monomer such as hydrochinonmonomethylether, hydrochinondimethylether, BHT and/or a stabilizer that stabilizes anionic polymerizable monomers such as acids like phosphoric acid, arylphosphonic acid, SO₂, p-toluensulfonic acid.

13. A dental composition of claim 1 that is usable as dental filling material, dental cement, dental sealer or as dental adhesive.
